SEQUENCE LISTING (11 sequences)

SEQ ID NO: 1 - protein, 10 aa
  Modified-site 1: /product= “OTHER” /note= “Xaa is Lys or His”
  Modified-site 3: /product= “OTHER” /note= “Xaa is Lys or Gly”
  Modified-site 4: /product= “OTHER” /note= “Xaa is Glu or Asn”
  Modified-site 6: /product= “OTHER” /note= “Xaa is Leu or Arg”
  Modified-site 8: /product= “OTHER” /note= “Xaa is Gln or Pro”
  Modified-site 10: /product= “OTHER” /note= “Xaa is Glu or Leu”
XXXXLXAXXX

SEQ ID NO: 2 - protein, 10 aa
XXXPQVLNYK

SEQ ID NO: 3 - protein, 10 aa
MQLKPMEINP

SEQ ID NO: 4 - protein, 10 aa
KHSLPDLPYD

SEQ ID NO: 5 - protein, 10 aa
  Modified-site 1: /product= “OTHER” /note= “Xaa is His or Asp or Ser or Gln”
  Modified-site 5: /product= “OTHER” /note= “Xaa is Glu or Gln”
  Modified-site 7: /product= “OTHER” /note= “Xaa is Arg or Ile or Leu”
  Modified-site 9: /product= “OTHER” /note= “Xaa is Lys or Ala or Arg”
  Modified-site 10: /product= “OTHER” /note= “Xaa is Gln or Arg”
XELPXVXDXX

SEQ ID NO: 6 - protein, 10 aa
XXAPLTATAP

SEQ ID NO: 7 - protein, 10 aa
  Modified-site 1: /product= “OTHER” /note= “Xaa is Ser or Asp or Gly”
XXVLLMKYLG

SEQ ID NO: 8 - protein, 10 aa
  Modified-site 1: /product= “OTHER” /note= “Xaa is Gly or Ile or Lys or Met”
  Modified-site 2: /product= “OTHER” /note= “Xaa is Glu or Arg”
  Modified-site 3: /product= “OTHER” /note= “Xaa is Val or Leu”
  Modified-site 4: /product= “OTHER” /note= “Xaa is Lys or Gln or Thr or Glu”
  Modified-site 5: /product= “OTHER” /note= “Xaa is Val or Gln”
  Modified-site 6: /product= “OTHER” /note= “Xaa is Asp or Phe or Leu”
  Modified-site 9: /product= “OTHER” /note= “Xaa is Arg or Ile”
  Modified-site 10: /product= “OTHER” /note= “Xaa is Gln or Lys or Phe or Ile”
XXXXXXMAXX

SEQ ID NO: 9 - protein, 10 aa
MLTELEKALN

SEQ ID NO: 10 - protein, 10 aa
TTSIRQFTSS

SEQ ID NO: 11 - protein, 9 aa
XTXILKFTL

(19) United States
(12) Patent Application Publication
(10) Pub. No.: US 2005/0095249 A1
(43) Pub. Date: May 5, 2005

(54) Protein markers for lung cancer and use thereof

(75) Inventor: Samir M. Hanash, Ann Arbor, MI (US)

Correspondence Address: ARENT FOX KINTNER PLOTKIN & KAHN, 1050 CONNECTICUT AVENUE, N.W., SUITE 400, WASHINGTON, DC, 20036, US

(73) Assignee: The Regents of the University of Michigan

(21) Appl. No.: 10/461,424

(22) Filed: Jun. 16, 2003

Related U.S. Application Data
(63) Continuation of application No. 09/022,527, filed on Feb. 12, 1998, now abandoned.
(60) Provisional application No. 60/038,819, filed on Feb. 12, 1997.

(51) Int. Cl. G01N 33/574; C12Q 1/68; A61K 39/395; C07K 14/705; C07K 16/30

(52) U.S. Cl. 424/155.1; 435/7.23; 530/388.8; 530/350

(57) ABSTRACT

Computerized analysis of 2-D gels, both carrier ampholyte (CA) and immobilized pH gradient (IPG) based, of the proteins in tissue from lung tumors, reveals proteins which are different in different types of tumors and in control tissues.

This application claims the benefit of U.S. Provisional Application No. 60/038,819, filed Feb. 12, 1997.

### BACKGROUND OF THE INVENTION

1. Field of the Invention

The present invention relates to proteins which are markers for lung cancer.

A large number of polypeptides that are differentially expressed between the three major lung tumor types have been identified. A small number of these polypeptides overlap with markers previously identified as markers for esophageal tumors. However, the majority (some thirty polypeptides) are new to the present analysis.

2. Description of Related Art

Lung cancer is the major cause of cancer deaths in men over 35 years of age and is a leading cause of death in women in this age group. There are several sub-types of lung cancer. Squamous cell carcinoma, adenocarcinoma and small cell carcinoma represent major sub-types. In view of the overall high incidence and mortality of lung cancer, approaches to screen and detect this type of cancer at an early stage would be quite beneficial. However the benefits of currently available screening strategies are doubtful and there remains much need for more effective strategies. To that effect, the identification of biochemical markers with a high degree of specificity for tumors and specific subtypes of tumors would be beneficial.

At the present time, lung cancer is diagnosed primarily by biopsy. Unfortunately, by the time the cancer is diagnosed it is often far advanced. Survival after diagnosis is poor.

Thus, a need exists for the diagnosis of lung cancer at an early stage. Markers which correspond to the advance of the illness may be used to monitor therapeutic regimens.

### SUMMARY OF THE INVENTION

The strategy of the present invention involves analyzing several hundred cellular proteins expressed in different lung cancer sub-types to identify proteins that are subtypes(s) specific. Using the procedure of two-dimensional gel electrophoresis, a subset of proteins that appear to distinguish between the major sub-types in a statistically significant manner has been detected. These proteins have utilities in many areas, including the following:

1. Screening normal individuals or individuals at an increased risk for lung cancer.

2. Establishing the specific lung cancer sub-type at the time of diagnosis.

3. Providing an indication of prognosis for individuals diagnosed with a specific lung cancer sub-type.

4. Providing novel approaches for therapy, based on understanding of the role of these proteins in different lung cancer sub-types.

By comparison of 2-D gels showing proteins from normal lung and different types of lung tumors such as squamous, small cell, and adenocarcinoma, a set of proteins have been identified in the different source tissues. These proteins provide information on the pathogenesis of lung tumors, and have utility as markers to monitor therapeutic regimens. The proteins can also be purified and used as immunogens to generate antibodies which can be used as diagnostic reagents. In addition, some of the proteins or antibodies thereto may have therapeutic applications.

### BRIEF DESCRIPTION OF THE DRAWINGS

FIG. 1 shows an Isoelectric-Focusing (IEF) gel of a sample from a patient with Squamous cell lung cancer.

FIG. 2 shows an IEF gel of a sample from a patient with classical small cell lung cancer.

FIG. 3 shows an IEF gel of a sample from a patient with adenocarcinoma of the lung.

### DETAILED DESCRIPTION OF THE INVENTION

One aspect of the invention is a new diagnostic method for lung tumors. The diagnostic method is based on the detection of at least one protein which is overexpressed in lung tumors relative to non-tumor lung tissues and which is specific for a lung tumor sub-type. In order to identify the protein(s) to be used in lung tumor diagnosis, proteins expressed in 60 lung tumors were analyzed using 2-D gel electrophoresis. By comparing the protein gel electrophoresis profiles of lung tumors and non-tumor lung tissues, proteins which are overexpressed in lung tumors were located. As demonstrated below, some of the specific proteins over-expressed also correlate with the lung tumor sub-type. Therefore, by concentrating on a plurality of protein markers which are overexpressed in different specific lung tumor subtypes, a diagnosis of the lung tumor sub-type can be made. For instance, relying on at least three protein markers each specific for one of three major lung tumor subtypes, i.e. squamous cell carcinoma, adenocarcinoma or small cell carcinoma, a diagnosis of the major lung tumor subtype can be made. It should be emphasized that the protein markers can be determined using gel electrophoresis in the absence of antibodies, an immunoassay if antibodies specific for the protein markers are available or any other method of detecting the protein markers. Antibodies specific for the protein markers allow in vitro or in vivo applications of the diagnostic method.

Another aspect of the invention is a method to monitor the progress of treatment of lung tumors by monitoring the appearance of at least one specific protein marker for the lung tumor sub-type being treated. Some of the protein markers identified in the instant invention can be monitored during the course of treatment of a lung tumor with an emphasis on the protein markers specific for the lung tumor sub-type under treatment. As the treatment progresses, the presence of at least one of these specific protein markers can be followed as another way to judge the treatment effectiveness.

Carrier Ampholyte (CA) Based 2-D Gels of Lung Cancer.

Tissue from over 60 lung tumors was obtained for 2-D analysis.

Most of the tumors have a pair of replicate silver stained gels available, in which the first dimension gel was an iso-electric focusing gel. In addition, most of the tumors were analyzed using immobilized pH gradients. The common tumor types are well represented: classical Small Cell (SC), Adenocarcinoma (Ad) and Squamous (Sq) tumors of the lung. Rarer tumor types were represented by fewer samples.

The analysis of the three main lung tumor types employed visual analysis of 3 large batches of gels that contained the largest numbers of the tumor types of interest (more than 10 of each of the three types). Images were also studied on the computer, one small close-up section at a time, matching those spots between images that appears to hold the most promise on a subset of the very best images. For the computerized analysis, spots were matched to image Ab6148, a SC sample, from which the “lung” spot numbering system used here is derived. This master is also matched to the master image used in the tumor studies including esophagus, colon, pancreas, leukemia, brain and breast tumors, so that each spot of interest in lung also has a spot number in the other systems. At the time spots of interest were identified, comments about each spot were made, largely concerning which samples had the largest or smallest spots.

It appears that certain sets of interesting spots should be treated as groups, that is, that they are likely to be the product of a single gene, differing only in their post-translational modification. This interpretation is based on the proximity of the spots on the gel, the geometry of the constellation that they form (e.g., a “charge chain”), their identical color with silver staining, and the fact that the quantities in different samples are correlated. In such cases, only a single spot has been selected for quantitation, typically the largest in the group or the spot that exhibits the least overlap with other spots thought to be unrelated. The groups and the representatives chosen for quantitation are:

| Group | Spot Quantitated |
|---|---|
| 37-40 | 40 |
| 28-30 | 29 |
| 52-54 | 53 |
| 33-35 | 33 |
| 87-89 | 87, 88 the P18 protein spots |

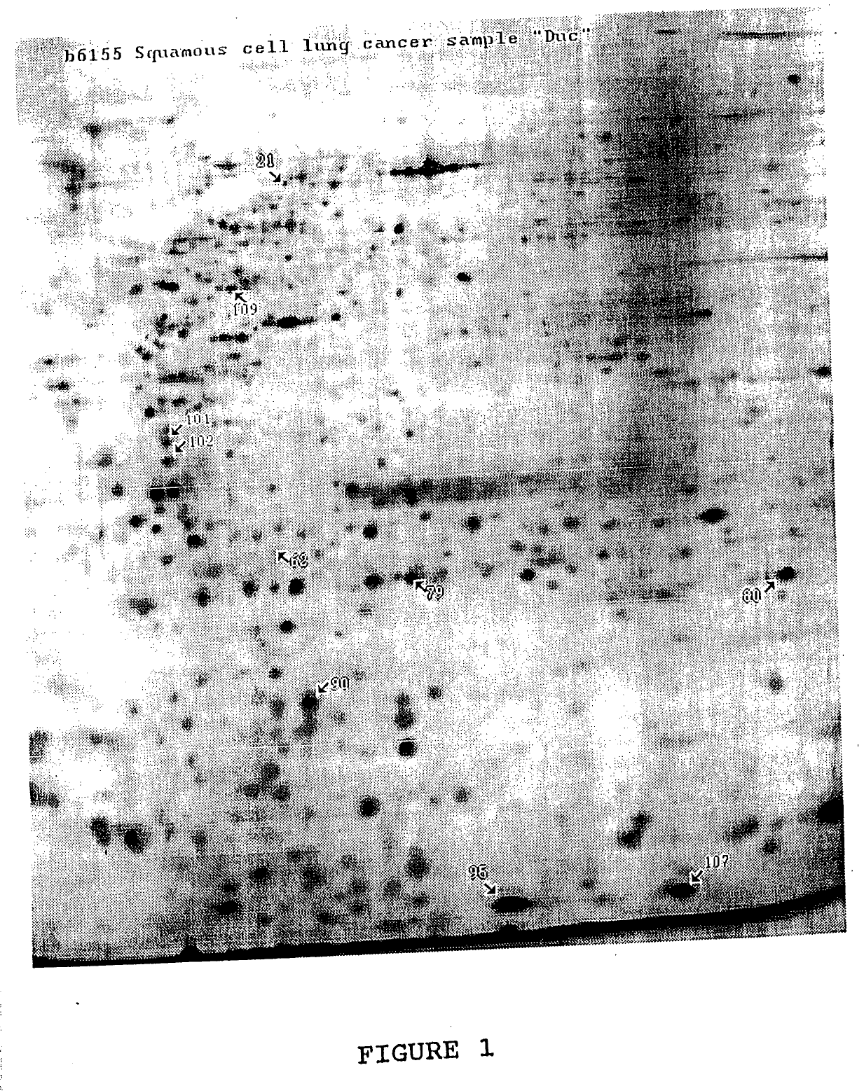

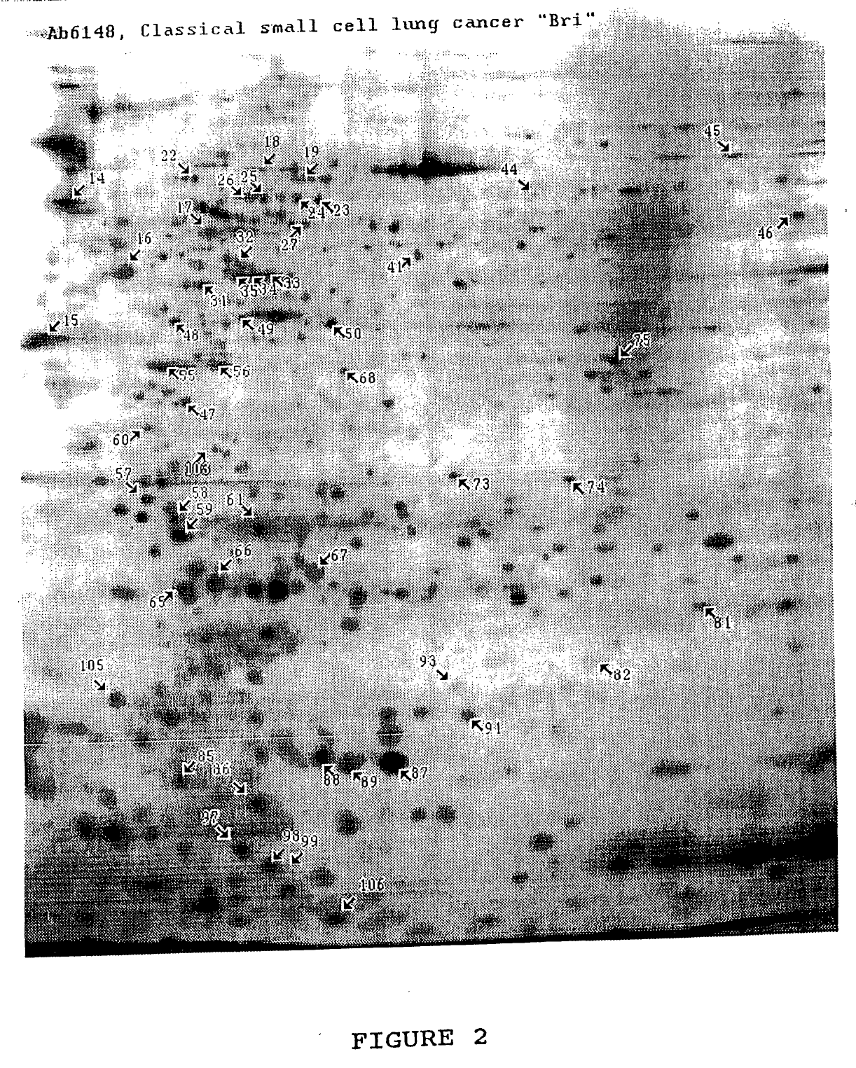

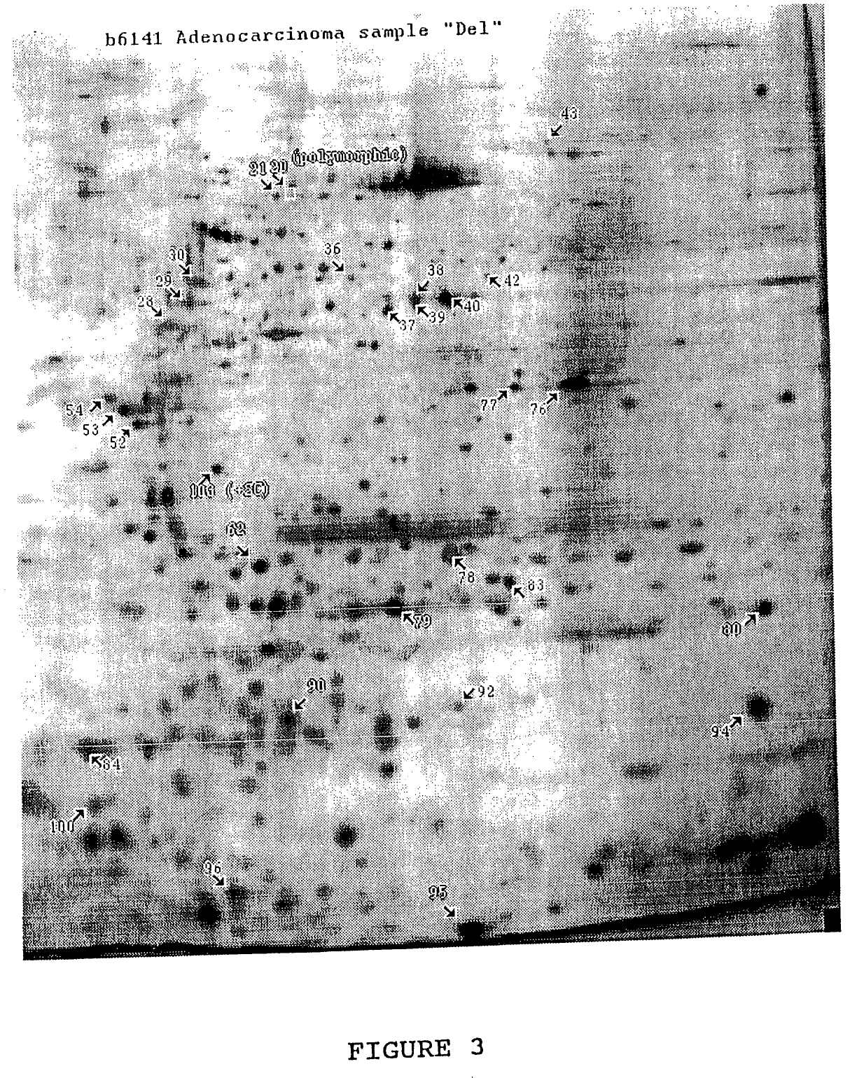

FIGS. 1-3 show the location of the candidate spots. These are labeled with spot numbers specific to the lung tumor matching.

Carrier ampholyte-based 2-D gels that cover the pH range of approximately 3.5-10.0 were prepared for all specimens.

Tissue was solubilized by addition of lysis buffer consisting of (per liter) 8 M urea, 20 ml of Nonidet P40 surfactant, 20 ml of ampholytes (pH 3.5-10), 20 ml of 2-mercaptoethanol, and 0.2 mM of phenylmethylsulfonyl fluoride in distilled deionized water. Approximately 30 μl aliquots containing 70 μg of protein were loaded on individual gels.

Because isoelectric focusing is sensitive to charge modification, it is important to minimize protein alterations (e.g., proteolysis, deamidation of glutamine and asparagine, oxidation of cystine to cystic acid, carbamylation) that can result from improper sample preparation. Once solubilized, samples may be stored frozen at −80° C. for short periods (<1 month) without significant protein modification). 2-D PAGE was done as previously described (Strahler et al, Journal of Clinical Investigation, 85:200-207, 1990). In most cases aliquots were immediately applied onto isofocusing gels. First-dimension gels contained 50 ml of ampholytes per liter (pH 3.5-10). Isofocusing was done at 1,200 V for 16 h and 1,500 V for the last 2 h. 20 gels were run simultaneously. For the second-dimension separation, an acrylamide gradient of 11.4-14.0 g/dl was used. Protein spots in gels were visualized by the silver-staining technique of Merril et al. (Merril et al, Science, 211:1437-1438, 1981).

Immobilized pH Gradient (IPG) 2-D Gels of Lung Cancer

In addition to generating 2-D patterns that were carrier ampholyte-based, a second set of patterns using immobilized pH gradients were generated for many of the tumors.

Samples were prepared as for the CA based 2-D gels of lung cancer discussed above. For first dimension separation an immobilized pH gradient covering the separation range of pH 4-10. The second dimension is the same as for the CA based 2-D gels.

IPG gels are prepared using derivatives of acrylamide having carboxyl or tertiary amino groups with specific pK values. A linear pH gradient is prepared from a dense, acidic solution and a light, basic solution using a two-chamber microgradient former. The pH gradient is stabilized during polymerization of the Immobiline-acryl-amide-bisacrylamide matrix by a co-linear gradient of glycerol. Formulations of buffering Immobiline mixtures with titrating Immobiline for the pH limit solutions for narrow pH gradients (1 pH unit) or for broad pH gradients (>1 pH unit, up to 6 pH units) (Gianazza et al, Electrophoresis 6:113 (1985) and LKB application Note 324 (1984)) have been published.

The second dimension separates proteins on the basis of molecular weight in an SDS gel. An 11.5 to 14% T (2.6% cross-linking) acrylamide gradient provides effective separation of proteins of mass from 15,000 to 100,000. Proteins outside this range are less well resolved. Proteins with molecular weight less than 10,000 Da electrophorese close to the dye front and are not resolved.

Computer Assisted Analysis of 2-D Gels

Each gel was scanned in a 1024×1024 pixel format, where each pixel can have one of 256 possible values representing different degrees of intensity. Spot lists for study images are matched to spot lists of master images so that the result is a hierarchy of matched protein spots. The purpose of the matching is to link the same polypeptide spot through the hierarchy to allow assessment of its presence, quantitative variation and specificity, as described in Strahler et al., 1990. For comparison of the amount of individual proteins between gels, an adjustment process is utilized. The integrated intensity of detected polypeptides, measured in units of optical density per square millimeter, is adjusted relative to the intensity of reference polypeptides that are ubiquitously expressed. The adjustment is made to compensate for any variation between gels due to protein loading or staining.

Most spots of interest were quantitated and the results are shown in Tables 1-5. A few spots that appear in FIGS. 1-3 as interesting do not appear in the Tables. Factors for not including spots are:
They are part of a larger family of spots as explained above.
Interest in them diminished after the quantitation results were analyzed (e.g., lung 32, 44, 46, 99).
They have been studied previously. This includes lung spot numbers 23-26 (np65's), 56 (B23's), 87-89 (P18's), 97 (CRBP-1), 60 (PCNA), 78 (Hsp27), as well as NDPK-A. A few of these famous spots were quantitated to help characterize each tumor sample (P18, P18a, CRBP-I, Hsp27, Hsp27a).
   Assessment of Spots in Other Tissues

A variety of normal tissues and tumors have been studied in an effort to gain some insight into the spots found interesting in lung tumors. The spots included in the list below represent that subset of spots that were quantitated and are considered very interesting. Some quantitated spots are considered less interesting at this time because the differences between lung tumors were not statistically very significant, the mean differences between tumor types were not very large, or because the spots did not appear very much larger in tumors than in control lung samples.

Some spots are still included even though they did not give very small P-values. Usually this is because it is believed that there is potentially an interesting difference, but the fairly simple statistical tests employed are ignoring group (gel batch) effects or are affected by a few cases where the samples do not all agree perfectly (inflated variance measures). It was also in a spot's favor if it had been identified as interesting in previous studies, including studies of esophagus tumors.

GELS:

Brain: Medulloblastoma, Glioblastoma, and normal samples.
Breast Tumors.
Leukemias: AML=ANLL, CALL and normal PBL's
Lung Tumors: Squamous (Squ), Small Cell (SC), Adenocarcinomas (Adn) and normal lung samples (NM).
Neuroblastomas: Various stages and myc copy numbers.
Esophagus: Squamous Carcinomas of the Esophagus (SC), normal esophagus (NE), gastric mucosa (GM), Barrett's (BA), esophageal adenocarcinoma (EA) and tumor of the cardia (TC).
   Entries:
L=Large, as big or bigger than in Esophageal adenocarcinoma or Tumor of the Cardia.
M=Medium, there but not as big as in tumors of interest.
S=Small
A=Absent

S? or A? indicates inability to identify the spot in some tissue, simply because there is nothing like what was seen in the tumors in the area. Conversely L? means there is a big spot in the location, but it is uncertain whether it is the sample spot. A * indicates that there is a note below.

The first spot numbers are those used in matching lung tumors (Ab6148). The second spot numbers are from the master image from esophagus (Bb9779). A “@” by esophagus indicates that the spot was noted as interesting in that esophageal tissue. There are sometimes notes for these spots in esophagus samples in other reports. One general observation is that it is easiest to compare SC lung with neuroblastomas.

The first block of spots was initially thought to be larger in SQ or Ad lung (usually Ad) while the second block of spots was thought larger in SC lung samples. The quantitative results should be used to judge the exact status with regard to spot sizes in the different sample types, since sometimes a spot is larger in two of the types, or has a pattern of being largest in one type, smallest in another, and intermediate in the third tumor type.

Spot Quantitation for Lung Tumors.

Spots in digital images of Lung Squamous tumors (Sq), Adenocarcinoma tumors (Ad), and Small Cell Lung cancers (SC) from 3 runs of IEF gels were quantitated. There were 9 Sq, 8 Ad and 9 SC samples in total. Sources of the samples were primary tumors (PT) or metastatic (MT). The groups of gels formed by electrophoretic runs are labeled, A, B and C in the first column of the table. “Stage” of the tumor is labeled under “stg”.

The gels with images matched to a master lung pattern were largely those from the group labeled “A”. Some spots were omitted because they are difficult to quantitate, because they seem to be a member of a family of spots only one of which appears in the table below, or because they are already known. Ten reference spots that appear to be more or less invariable between sample types were also quantitated, for use in adjusting the spot integrated intensity data. The spots are labeled in the order of another table in which other tissue types were surveyed. Four “famous spots” (L2=phosphorylated Hsp27, L4=unphosphorylated Hsp27, P18 and P18a phosphorylated P18) are also included to help characterize the samples.

Gel to gel adjustment using the ten reference spots was by what has become the usual method. A standard was formed by computing the average size of each spot across the gels in this study. To compute the adjustment for a particular gel, the ratios of each spot on the gel to the standard were calculated and the ratios were averaged (by taking antilogarithms of the average log ratio). Raw spot integrated intensities are divided by this adjustment factor to obtain the adjusted integrated intensities tabled below. For each gel the adjustment factor is tabled under “Dark”.

For each spot the means and variances with each sample type are given as well as the p-value for an F-test of whether the 3 means are identical. There appear to be run effects and individual effects for some spots, which should probably be judged by eye, and this run effect is why the data is tabled in blocks according to groups formed by electrophoretic runs Often one can see that the significance for tests considering group effects would be greater, or that omitting a single individual with an enormous value would reduce the variances enough to change the P-value considerably.

Potential Markers

14. Occurs as a large spot in small cell lung cancer. It is absent in normal lung tissue and occurs as a small spot in adenocarcinoma of the lung and in squamous cell lung cancer. In most other tissues and cancers with the exception of medulloblastoma where it is large, it occurs as a small intensity spot.

15. Has a similar intensity and tissue distribution pattern as spot 14. It is likely to represent a group of related polypeptides which are not separated.

16. Occurs as a medium intensity spot in small cell lung cancer. It is present in small amounts in normal lung tissue and occurs as a small spot in adenocarcinoma of the lung and in squamous cell lung cancer. In most other tissues and cancers with the exception of medulloblastoma where it is large, it is either absent or occurs as a small intensity spot.

17. Occurs as a large intensity spot in small cell lung cancer. It is present in small amounts in normal lung tissue and occurs as a small spot in adenocarcinoma of the lung and in squamous cell lung cancer. In most other tissues and cancers with the exception of medulloblastoma where it is large, it is either absent or occurs as a small intensity spot.

22. Occurs as a moderate intensity spot in small cell lung cancer. It is present in smaller amounts in normal lung tissue and occurs as a small spot in adenocarcinoma of the lung and in squamous cell lung cancer. In most other tissues and cancers it is either absent or occurs as a small intensity spot.

27. Occurs as a moderate intensity spot in small cell lung cancer. It is absent in normal lung tissue and occurs as a smaller spot in adenocarcinoma of the lung and in squamous cell lung cancer. In most other tissues and cancers it is either absent or occurs as a small intensity spot.

31. It is a moderate to large intensity spot in small cell lung cancer. It is absent in normal lung tissue and occurs as a smaller spot in adenocarcinoma of the lung and in squamous cell lung cancer. In most other tissues and cancers it is either absent or occurs as a small intensity spot.

33. Occurs as a moderate intensity spot in small cell lung cancer. It is absent in normal lung tissue and in adenocarcinoma of the lung and in squamous cell lung cancer. In most other tissues and cancers it is either absent or occurs as a small intensity spot.

50. Occurs as a prominent spot in small cell lung cancer and occurs as a small spot in normal lung and in adenocarcinoma of the lung and in squamous cell lung cancer. In most other tissues and cancers with the exception of brain and some brain tumors where it is large, it occurs as a moderate to small intensity spot.

68. Occurs as a moderate size spot in small cell lung cancer and occurs as a smaller spot in normal lung and in adenocarcinoma of the lung and in squamous cell lung cancer. In most other tissues and cancers with the exception of brain and some brain tumors where it is large, it occurs as a moderate to small intensity spot.

47. Occurs as a large intensity spot in small cell lung cancer. It is absent in normal lung tissue and occurs as a small spot in adenocarcinoma of the lung and in squamous cell lung cancer. In most other tissues and cancers, it occurs as a small intensity spot.

57. Occurs as a moderate intensity spot in small cell lung cancer. It is smaller in normal lung tissue and in adenocarcinoma of the lung and in squamous cell lung cancer. In most other tissues and cancers, it occurs as a small intensity spot.

58. Occurs a large intensity spot in small cell lung cancer. It is smaller in normal lung tissue and in adenocarcinoma of the lung and in squamous cell lung cancer. In most other tissues and cancers, with the exception of brain in which it is large, it occurs as a small to moderate intensity spot.

59. Occurs as large intensity spot in small cell lung cancer and in esophageal adenocarcinoma. It is smaller in normal lung tissue and in adenocarcinoma of the lung and in squamous cell lung cancer. In most other tissues and cancers, with the exception of brain in which it is large, it occurs as a small to moderate intensity spot.

61. It is a moderate to large intensity spot in small cell lung cancer. It is absent in normal lung tissue and occurs as a smaller spot in adenocarcinoma of the lung in squamous cell lung cancer. In most other tissues and cancers it is either absent or occurs as a small intensity spot.

66. It is a moderate to large intensity spot in small cell lung cancer. It is absent in normal lung tissue and occurs as a smaller spot in adenocarcinoma of the lung and in squamous cell lung cancer. In most other tissues and cancers it is either absent or occurs as a small intensity spot.

67. Occurs as a large spot in small cell lung cancer. It is absent in normal lung tissue and occurs as a small spot in adenocarcinoma of the lung and in squamous cell lung cancer. In most other tissues and cancers except brain related, in which it is large, it is either absent or occurs as a small intensity spot.

73. Occurs as a moderate intensity spot in small cell lung cancer. It is absent or small in normal lung tissue and occurs as a small spot in adenocarcinoma of the lung and in squamous cell lung cancer. In most other tissues and cancers except brain related, in which it is moderate, it is either absent or occurs as a small intensity spot.

74. May be related to 73. Occurs as a moderate intensity spot in small cell lung cancer. It is absent or small in normal lung tissue and occurs as a small spot in adenocarcinoma of the lung and in squamous cell lung cancer. In most other tissues and cancers except brain related in which it is moderate, it is either absent or occurs as a small intensity spot.

81. It is a large intensity spot in small cell lung cancer. It is absent in normal lung tissue and occurs as a smaller spot in adenocarcinoma of the lung and in squamous cell lung cancer. In most other tissues and cancers it is either absent or occurs as a small intensity spot.

105. It is a moderate to large intensity spot in small cell lung cancer. It is small in normal lung tissue and in adenocarcinoma of the lung and in squamous cell lung cancer. In most other tissues and cancers it is either absent or occurs as a small intensity spot.

86. It is a large intensity spot in small cell lung cancer. It is small in normal lung tissue and in adenocarcinoma of the lung and in squamous cell lung cancer. In most other tissues and cancers it is either absent or occurs as a small intensity spot.

97. It is a large intensity spot in small cell lung cancer. It is absent in normal lung tissue and small in adenocarcinoma of the lung and in squamous cell lung cancer. In most other tissues and cancers it is either absent or occurs as a small intensity spot.

98. It is a moderate to large intensity spot in small cell lung cancer. It is absent in normal lung tissue and occurs as a smaller spot in adenocarcinoma of the lung and in squamous cell lung cancer. In most other tissues and cancers it is either absent or occurs as a small intensity spot.

106. It is a moderate to large intensity spot in small cell lung cancer. It is absent in normal lung tissue and occurs as a smaller spot in adenocarcinoma of the lung and in squamous cell lung cancer. In most other tissues and cancers it is either absent or occurs as a small intensity spot.

109. Occurs as a moderate intensity spot in squamous cell lung cancer and is absent in normal lung and either absent or occurs as a small size spot in other cancers with the exception of squamous esophageal cancer in which it is large.

101. Occurs as a moderate intensity spot in squamous cell lung cancer and lung adenocarcinoma and is small in normal lung tissue. It is either absent or occurs as a small size spot in other cancers with the exception of squamous esophageal cancer in which it is large.

102. Has a similar pattern of expression as 101.

107. Occurs as a large intensity spot in squamous cell lung cancer and adenocarcinoma and is absent or small in normal lung tissue. It is small in small cell lung cancer and moderate to large in a number of other cancers.

21. Occurs as a large intensity spot in squamous cell lung cancer and adenocarcinoma and is moderate in normal lung and small in small cell lung cancer. It is also large in squamous and adenocarciroma of the esophagus and occurs in variable size in other cancers.

62. Occurs as a large intensity spot in squamous cell lung cancer and adenocarcinoma and is moderate in normal lung and small in small cell lung cancer. It occurs in variable size in other cancers.

79. Occurs as a large intensity spot in squamous cell lung cancer and adenocarcinoma and is large in normal lung. It is small in small cell lung cancer. It occurs in variable size in other tissues and cancers.

80. Occurs as a large intensity spot in squamous cell lung cancer and adenocarcinoma and is small to moderate in small cell lung cancer. It is difficult to detect or absent in most other tissues.

90. Occurs as a large intensity spot in squamous cell lung cancer and adenocarcinoma and is small to moderate in small cell lung cancer and small in normal lung. It is variable in other tissues and cancers.

95. Occurs as a large spot near the dye front in squamous cell lung cancer and adenocarcinoma and it is small to moderate in small cell lung cancer and small in normal lung tissue. It is variable or undetectable in other tissues and cancers.

43. Occurs as a large intensity spot in squamous cell lung cancer and adenocarcinoma and is small to moderate in small cell lung cancer. It is difficult to detect or absent in most other tissues.

29. Occurs as a large spot in squamous cell lung cancer and adenocarcinoma and is small to moderate in small cell lung cancer and normal lung tissue. It is variable in most other tissues.

40. Occurs as a large spot in squamous cell lung cancer and adenocarcinoma and is small to moderate in small cell lung cancer and normal lung tissue. It is variable in most other tissues.

42. It is an inconspicuous spot that is most prominent in squamous cell lung cancer and adenocarcinoma and is smaller or absent in most other tissues.

53. Part of a train of spots that is prominent in lung adenocarcinoma.

83. This spot is prominent in squamous cell lung cancer and adenocarcinoma and it is smaller or absent in most other tissues.

92. It is an inconspicuous spot that is most prominent in squamous cell lung cancer and adenocarcinoma and it is quite small or absent in most other tissues.

94. This spot is prominent in squamous cell lung cancer and adenocarcinoma and it is difficult to detect or absent in most other tissues.

84. This spot is most prominent in lung and esophageal adenocarcinoma and squamous cell cancer and is variable in other tissues and cancers.

100. It is an inconspicuous spot that is most prominent in squamous cell lung cancer and adenocarcinoma and it is quite small or absent in most other tissues.

96. Occurs as a large intensity spot in squamous cell lung cancer and adenocarcinoma and is small to moderate in small cell lung cancer and normal lung tissue. It is variable in most other tissues.

Antibody Production

The proteins eluted from the gels, or peptide fragments thereof, may be used as immunogen for the production of antibodies. The antibodies may be polyclonal antibodies or may be monoclonal antibodies. The antibodies are made by methods known to those skilled in the art. Antibodies with very high affinity and specificity may be used for immunological tests for markers of cancer.

For the production of polyclonal antibodies, the immunogen, usually mixed with an adjuvant, is injected into a host animal, such as a mouse, guinea pig, rabbit, goat or horse. The injection is repeated at the same site or different sites at regular or irregular intervals. The host animal is bled periodically to assess antibody titer until it is determined that optimal titer has been reached. The antibodies are obtained either from antiserum taken from the host animal with bleeding or by somatic cell hybridization techniques known in the art.

Monoclonal antibodies can be produced by a method known in the art, e.g. Kohler and Milstein (Nature, vol. 256, pp. 495-497, 1975). Generally, spleen cells are obtained from a host animal injected with the immunogen or a fragment thereof. The spleen cells are immortalized by fusion with an immortal cell line, preferably a myeloma cell line, of the same or different species as the injected host animal. The fused cells are cloned and the resulting hybridomas are screened for production of monoclonal antibodies that specifically bind the immunogen.

In the instant application, the term “an immunological assay” means any method known in the immunology art for the quantitation of substances. An example of an immunological assay is radioimmunoassay.

In Vivo Applications

The antibodies produced may be conjugated with a radioactive tag and injected into a patient. With appropriate imaging techniques the tumor can be located using the radioactively conjugated antibody. If the amount of radioactivity attached to the antibody is increased considerably, or the antibody is conjugated to a toxin or an anti-tumor drug, the conjugate can be used to kill tumor cells in vivo. The antibody provides the targeting function, and the toxin, anti-tumor drug or radioactivity kills the cells which are targeted by the antibody. The radioactive tag can be any isotope giving off alpha particles, beta particles or gamma rays. The toxin can be any substance, such as ricin, known to be toxic to cells. The anti-tumor drug includes any drug, e.g. daunorubicin, 5-fluorouracil, or derivatives thereof, or methotrexate, effective in treating tumors. Using an antibody conjugated with radioactivity, a toxin or drug for tumor therapy is known in the art, for instance see Roitt, I. et al, Immunology, pp. 20.8 and 20.9, Mosby, London, 1996, which is incorporated by reference. An effective dose can be 0.005 to 500 mg antibody per kg body weight. The conjugate can be administered by intravenous, intramuscular or subcutaneous injections.

The protein markers can also be used in immunotherapy of lung tumors. For instance, immunocompetent cells from the blood of a patient can be repeatedly exposed in vitro to one or more protein markers specific for the sub-type of lung tumor that the patient has. The challenged immunocompetent cells can later be injected into the same patient for immunotherapy of the lung tumor.

Gene Therapy

The gene corresponding to tumor specific proteins identified by the method of the present invention may be isolated and identified. Methods to isolate the gene corresponding to a given protein are well known to those skilled in the art. The gene can then be inactivated by molecular biological techniques and replaced into the body by gene therapy. Alternatively, anti-sense molecules can be made to genes of the tumor specific markers, and the anti-sense molecules can be used as therapeutics. By either of the above methods known to those skilled in the art, the tumor specific gene expression is decreased.

Studies of MRP8 and MRP14 and of their Relevance to Tumor Biology and as Tumor Markers

In studies comparing 2D protein patterns from various types of lung tumors (i.e., Squamous cell carcinoma, Adenocarcinoma and Small cell carcinoma) a protein spot was identified in these tumor types which was found to be absent in the patient's normal lung tissue. This protein gave the sequence MLTELEKALN, which is 100% homologous with human MRP-8. Further, on the 2D protein patterns for lung tumors having a large MRP-8 spot, the presence of an additional low molecular weight pair of spots was noted consistent with the two forms of MRP-14 (MRP14 has two translation initiation sites situated 4 codons apart), as determined by comparison with published figures. Among the spot proteins overexpressed in lung tumors, the preferred spot proteins are MRP8 and MRP14.

Relationship of MRP8 and MRP14 to Tumor Biology

MRP8 (IO kDa) and MRP 14 (14 kDa) are both calcium binding proteins which belong to the S 100 family of EF-hand proteins, a family which consists of at least 17 members. Of interest, genes for this family of proteins have been localized to human chromosome Iq2I, a region of the chromosome which is frequently rearranged in different tumor types. These proteins are proposed to play a role during differentiation, regulation of the cell cycle and cytoskeletal/membrane interactions. Both of these proteins are composed of two distinct EF-hands flanked by hydrophobic regions at either terminus and separated by a central hinge region. MRP8 has been demonstrated to mediate chemotactic activity on macrophages. Interestingly, a peptide encoded by the hinge region (between the two EFhands) has been shown to specifically mediate this effect. As such, these proteins might play a role in diseases which cause chronic inflammation, including cancer.

Both the N-terminal and carboxy-terminal EF-hands are able to bind calcium, although the carboxy-terminal EF-hand does have a higher affinity. MRP8 and MRP14 have both been shown to be secreted from granulocytes and monocytes. It is presently unclear how these proteins are secreted as they do not possess a classical signal peptide. One possibility is that calcium binding may expose a hydrophobic domain which could allow an interaction with the membrane, thereby resulting in secretion of the molecules. It has been demonstrated that both MRP8 and MRP14 homodimerize and heterodimerize with each other, thus forming complexes of various molecular weights. It is presently unclear as to the precise function of each homodimer and heterodimer form.

An antibody against the cystic fibrosis antigen (an epitope formed by heterodimerization of MRP8 and MRP14) also will react positively against a 14 kDa antigen which has been shown to be MRP14. The antibody is available commercially. This antibody has been utilized for immunohistochemistry on sections of tumor tissue and corresponding normal tissue from the same patient. These stained tissue sections revealed minimal staining in the normal lung tissue. There was somewhat more reactivity in the tumor tissue, most probably due to the increased presence of infiltrative cells. Of note, however, there was a very large amount of immunoreactivity in the area of normal tissue immediately adjacent to the tumor, thus suggesting that infiltrative cells (i.e., granulocytes, monocytes and/or macrophages) were being recruited to the tumor. Moreover, whether the antibody would recognize a specific 14 kDa protein in the serum of lung tumor patients was explored, at levels greater than that which might be present in the serum of normal individuals. The serum of 14 lung tumor patients and 14 normal individuals was separated by 1D electrophoresis, the proteins were transferred to PVDF membranes and probed with the commercial antibody. Integrated intensity analysis of reactivity in a band visualized at 14 kDa revealed markedly increased reactivity in the serum from tumor patients (n=14; mean intensity of 0.46) as compared to that in the serum from normal individuals (n=14; mean intensity of 0.09).

These findings indicate a role for antibodies against MRP in screening for different types of cancer in which the MRP's are detected in tumor tissue.

Sequencing

Amino acid sequencing of some of the above spot proteins was performed. The spots are eluted from the gels and subjected to sequence analysis. The amino acid sequences of some of the spot proteins are reported below. The correspondence of the spot protein and the Seq. ID No. is shown in the following table.

| Seq. ID No. | Spot Protein |
|---|---|
| 1 | 16 |
| 2 | 59 |
| 3 | 67 |
| 4 | 80 |
| 5 | 84 |
| 6 | 90 |
| 7 | 92 |
| 8 | 95 |
| 9 | 107 |
| 10 | 109 (major component) |
| 11 | 109 (minor component) |

Spot protein 109 has two components. The sequences of the major and minor components are listed in Seq. ID No. 10 and 11, respectively.

TABLE 1

| | | | | | | |
|---|---|---|---|---|---|---|
| sp# | sp# | Brain | Leukemia | Lung | esophagus samples | MW |

| | | | | | | | | | | | | | | | | | | | | | |
|---|---|---|---|---|---|---|---|---|---|---|---|---|---|---|---|---|---|---|---|---|---|
| lung | esop | Med | Gli | Nrm | Breast | AML | CLL | PBL | Squ | SC | Adn | NM | NBL | SC | NE | BA | EA | GM | TC | kD | PH |

| | | | | | | | | | | | | | | | | | | | | | |
|---|---|---|---|---|---|---|---|---|---|---|---|---|---|---|---|---|---|---|---|---|---|
| 109 | 92@ | A | A | A | A | A | A | A | M | A | S | A | A | L | S | A | A | A | A | 15 | 5.1 |
| 101 | 145@ | S | S | S | M | A | A | A | M | S | M | S | S | L | S | S | S | S | S | 35 | 4.2 |
| 102 | 146@ | S | S | S | M | A | A | A | M | S | M | S | S | L | S | S | S | S | S | 36 | 4.2 |
| 107 | 168 | A? | A? | A? | M? | ? | ? | ? | L | S | L | S? | S? | S? | L | M | M | M | M | 10 | 6.7 |
| 21 | 169 | S | S | S | L | L | L | L | L | S | L | M | S | L | S | M | L | M | L | 68 | 5.5 |
| 62 | 170 | S | S | S | S | ? | ? | ? | L | S | L | M | S | M | S | S | S | S | M | 27 | 5.4 |
| 79 | 120@ | S | L | L | L | S | S | S | L | M | L | L | M | M | M | L | L | M | M | 25 | 5.9 |
| 80 | 171? | L | L | M? | L? | ? | ? | ? | L | M | L | ? | ? | ? | ? | ? | ? | ? | ? | 25 | 7.1 |
| 90 | 124@ | ? | ? | ? | L? | ? | ? | ? | L | M | L | S | A? | M | S | L | M | A | A | 19 | 5.6 |
| 95 | 172 | A? | A? | A? | L? | A? | A? | A? | L | S | L | S | A | A? | L | S | M | M | M | 10 | 16.4 |
| 43 | 173 | S | M | S | S | A? | A? | A? | L | M | L | A? | A? | A | A | A | S? | A | A | 75 | 6.5 |
| 29 | 165@ | M | L | L | L | S | S | M | L | M | L | M | M | L | M | M | M | A | M | 49 | 4.6 |
| 40 | 111@ | M | L | L | S | A | A | A | L | M | L | M | M | S | M | L | L | M | M | 50 | 6.2 |
| 42 | 112@ | S | L | L | S | ? | ? | ? | L | S | L | M | S? | S | S | L | L | M | L | 53 | 6.3 |
| 53 | 174 | S | L | L? | M | A | A | A | M | S | L | M | L | S | S | M | M | S | S | 38 | 5.1 |
| 83 | 167@ | M | L | L | L? | A? | A? | A? | L | S | L | M? | M? | S | S | L | M | M | M | 26 | 4.5 |
| 92 | 175 | A | A | A | S | A | A | A? | M | S | M | A | A | A | A | A | A | A | A | 20 | 6.4 |
| 94 | 176? | A | A | A | ? | ? | ? | ? | L | M | L | ? | ? | ? | ? | ? | ? | ? | ? | 20 | 7.1 |
| 84 | 153@ | ? | ? | ? | M | S? | S? | S? | M | M | L | S | S? | L | S | M | L | S | M | 19 | 4.1 |
| 100 | 177 | A | A | A | M | A? | A? | A? | M | S | M | A | S? | A | A | A | S | A | S | 16 | 4.1 |
| 96 | 154@ | M | S | S | M | M | S | M | L | M | L | M | S | L | M | M | M | M | M | 11 | 5.2 |
| 14 | 178 | L | ? | ? | S | A | L? | A | S | L | S | A | M | S | S | S | S | S | S | 63 | 4.0 |
| 15 | 74 | L | S? | S? | L | S | L | M? | S | L | S | S | L | S | S | S | S | S | S | 43 | 3.9 |
| 16 | 157@ | M | L | S? | S | A | A | A | S | M | S | S | M | L | M | A | A | A | A | 52 | 4.2 |
| 17 | 179 | M | S? | S? | S | ? | ? | ? | S | L | S | S | L? | S | A? | S | S | A | S | 57 | 14.8 |
| 22 | 180 | M | M | S | S | A | A | S | M | L | M | S | M | L | S | S | S | S | S | 67 | 4.7 |
| 27 | 181 | M | S? | A? | A | S | S | S | S | L | S | A | M | S | A | S | M | A | S | 61 | 5.7 |
| 31 | 182 | A | A | A | M | ? | S | S | S | L | S | A | L | S | S | S | S | S | S | 50 | 14.8 |
| 33 | 183 | A? | A? | A? | A | ? | ? | ? | A | M | A | A | ? | A | A | A | A | A | A | 52 | 5.5 |
| 50 | 162@ | L | L | L | A | S | M | A | S | L | S | S | L | L | L! | M | M | M | S | 45 | 5.8 |
| 68 | 15 | L | L | M | S | L? | M | ? | M | L | M | S | M | S | M | M | M | L | M | 45 | 5.8 |
| 47 | 186 | S | S | S | M | S? | S? | S? | M | L | M | A | M | L | S | S | M | S | M | 37 | 4.5 |
| 57 | 187 | M | L | M | S | M | M | S | M | L | M | S | L | S | S | S | M | S | M | 31 | 4.2 |
| 58 | 188 | L | L | L? | M | M | S | S | M | L | M | M | M | M | S | S | M | S | S | 30 | 4.4 |
| 59 | 189 | L | L | L? | M | ? | ? | ? | M | L | M | M | L | M | M | M | L | M | M | 29 | 4.5 |
| 61 | 190 | M | M | ? | S | ? | ? | ? | S | L | S | A | M | A | S | A | A | A | S | 29 | 5.3 |
| 66 | 193 | S | S | S | M | ? | ? | ? | S | L | S | A | S | A | S | S | S* | S | S* | 25 | 4.5 |
| 67 | 194 | L* | L | L? | S | A? | A? | A? | S | L | S | A | L* | A | A | A | A | A | A | 26 | 5.8 |
| 73 | 195 | M | M | M | S? | ? | ? | ? | S | M | S | S? | L? | A | S? | S | S | S | S | 32 | 6.3 |
| 74 | 196 | M | M | M | S? | ? | ? | ? | S | M | S | A | L? | A | A | M | M | S | M | 32 | 6.7 |
| 81 | 197 | A | A | A | ? | A? | A? | A? | S | L | S | ? | A? | ? | A | A | A | A | S | 26 | 6.9 |
| 105 | 198 | A | A | A | M | M | M | S | M | L | M | S | L? | M | S | S | M | S | M | 20 | 4.1 |
| 86 | 199 | S | S | S | M | A? | A? | A? | S | L | S | S | M | S A | S | M | S | S | 15 | 4.9 | |
| 97 | 200 | M | A | A | A | A | M | S | S | L | S | A | L* | S* | A | A | A | A | A | 13 | 5.2 |
| 98 | 201 | S | ? | S | S | A | A | A | M | L | M | A | L | A | A | A | A | A | A | 12 | 5.5 |
| 106 | 202 | M | M | ? | ? | M? | M? | S? | M | L | M | A | M | A | A | S | S | A | A | 10 | 5.8 |

66: probably is the same as in lung. There is the possibility that a polymorphism here is confounding. If so it has the other allele to the right of 66 and lands at nearly the same postion as a common spot.
67: Very big in brain, neuroblastoma, and SC.
97: is CRBP-I. Neuroblastomas have it big, but not as big as SC.

TABLE 2

| group pat stg di | | | | | | | famous spots | | | | Initially thought large in squamous and/or adenocarcinoma | | | | | | | | | |
|---|---|---|---|---|---|---|---|---|---|---|---|---|---|---|---|---|---|---|---|---|
| gel ient | | | | ag so Dark | | | L2 | L4 | P18 | P18a | s109 | s101 | s102 | s107 | s21 | s62 | s79 | s80 | s90 | s95 |
| A | 155 | Duc | 1 | Sq | PT | 0.571 | 1.57 | 1.02 | 2.18 | 0.39 | 0.65 | 0.59 | 0.71 | 3.07 | 0.17 | 0.13 | 1.12 | 2.02 | 1.68 | 6.29 |
| A | 143 | Mor | 3 | Sq | PT | 0.485 | 0.57 | 0.46 | 0.48 | 0.04 | 0.08 | 1.14 | 1.14 | 0.48 | 0.32 | 0.17 | 2.43 | 1.87 | 1.77 | 2.68 |
| A | 156 | Chi | 3 | Sq | MT | 0.796 | 0.24 | 0.23 | 0.49 | 0.08 | 0.51 | 0.32 | 0.34 | 3.61 | 0.08 | 0.26 | 1.78 | 1.47 | 0.40 | 7.52 |
| B | 180 | Cay | 1 | Sq | UN | 1.681 | 2.07 | 0.90 | 0.46 | 0.47 | 0.59 | 0.14 | 0.12 | 1.57 | 0.20 | 0.90 | 3.44 | 1.26 | 0.42 | 3.06 |
| B | 171 | Wae | 3 | Sq | UN | 1.034 | 2.56 | 2.98 | 0.75 | 0.04 | 3.77 | 0.42 | 0.48 | 1.71 | 0.16 | 0.52 | 1.04 | 1.57 | 0.87 | 4.33 |
| B | 179 | Dur | 3 | Sq | PT | 1.326 | 2.29 | 2.29 | 0.48 | 0.05 | 0.35 | 0.28 | 0.48 | 0.80 | 0.17 | 0.37 | 1.72 | 1.68 | 0.23 | 4.45 |
| C | 225 | Arc | 2 | Sq | UN | 1.933 | 1.26 | 0.71 | 0.41 | 0.10 | 0.00 | 0.94 | 0.81 | 1.30 | 0.23 | 0.48 | 4.57 | 2.97 | 1.22 | 2.03 |
| C | 235 | Por | 2 | Sq | UN | 1.552 | 0.78 | 0.50 | 0.60 | 0.04 | 0.26 | 0.47 | 0.50 | 0.38 | 0.05 | 0.69 | 2.47 | 0.49 | 0.00 | 1.85 |
| C | 237 | Guy | 3 | Sq | PT | 0.947 | 2.98 | 2.93 | 0.35 | 0.06 | 1.99 | 0.33 | 0.47 | 3.66 | 0.15 | 0.14 | 2.91 | 1.55 | 0.43 | 3.19 |
| A | 141 | Del | 1 | Ad | PT | 0.837 | 0.98 | 1.98 | 1.41 | 0.13 | 0.00 | 0.13 | 0.37 | 0.27 | 0.24 | 2.09 | 3.55 | 2.00 | 1.81 | 5.65 |
| A | 150 | Bog | 2 | Ad | PT | 0.555 | 0.63 | 0.60 | 0.58 | 0.03 | 0.36 | 0.72 | 1.07 | 2.23 | -.-- | 0.24 | 4.72 | 4.03 | 1.28 | 3.94 |
| A | 144 | Des | 3 | Ad | PT | 0.567 | 0.88 | 2.10 | 2.46 | 0.27 | 0.00 | 0.20 | 0.13 | 1.00 | 0.11 | 0.40 | 2.66 | 1.20 | 0.53 | 3.21 |
| A | 158 | Suc | 3 | Ad | UN | 0.599 | 0.87 | 0.81 | 0.47 | 0.03 | 0.00 | 0.53 | 0.18 | 1.20 | -.-- | 0.31 | 4.28 | 2.93 | 1.71 | 1.89 |
| B | 167 | Coul | 3 | Ad | PT | 0.731 | 1.40 | 2.04 | 0.46 | 0.04 | 0.00 | 0.18 | 0.14 | 0.37 | -.-- | 0.21 | 1.49 | 1.57 | 0.69 | 0.74 |
| B | 172 | Bon | 3 | Ad | PT | 0.859 | 0.31 | 0.44 | 0.34 | 0.01 | 0.41 | 0.24 | 0.30 | 1.01 | 0.28 | 0.38 | 0.97 | 1.74 | 0.52 | 2.15 |
| C | 241 | Ber | 1 | Ad | UN | 0.797 | 1.35 | 0.83 | 2.12 | 0.22 | 0.00 | 2.13 | 2.07 | 0.72 | 0.08 | 0.51 | 4.56 | 1.94 | 0.00 | 7.52 |
| C | 234 | Dam | 3 | Ad | PT | 1.610 | 1.18 | 0.67 | 0.73 | 0.23 | 1.78 | 1.18 | 1.18 | 3.72 | 0.27 | 0.90 | 2.43 | 1.71 | 0.55 | 2.87 |
| A | 145 | Bai | 3 | SC | MT | 0.756 | 0.13 | 0.07 | 4.65 | 0.83 | 0.00 | 0.00 | 0.00 | 0.31 | 0.02 | 0.08 | 0.41 | 0.50 | 0.28 | 0.42 |
| A | 146 | Cha | 4 | SC | MT | 0.734 | 0.42 | 0.67 | 6.02 | 1.14 | 0.23 | 0.13 | 0.11 | 0.16 | 0.04 | 0.00 | 0.16 | 0.76 | 0.00 | 0.84 |
| A | 148 | Bri | 4 | SC | MT | 0.791 | 0.50 | 0.56 | 8.33 | 2.66 | 0.00 | 0.19 | 0.18 | 0.28 | 0.08 | 0.00 | 0.55 | 0.85 | 0.13 | 0.45 |
| A | 151 | Moy | 4 | SC | MT | 0.599 | 0.21 | 0.21 | 7.41 | 2.44 | 0.00 | 0.48 | 0.45 | 0.00 | 0.04 | 0.00 | 1.03 | 0.74 | 0.00 | 0.59 |
| A | 152 | Boua | 4 | SC | MT | 0.783 | 0.03 | 0.18 | 4.42 | 0.57 | 0.00 | 0.00 | 0.00 | 1.20 | 0.03 | 0.03 | 0.84 | 0.67 | 0.00 | 0.16 |
| A | 157 | Couc | 2 | SC | UN | 0.410 | 0.16 | 0.82 | 4.71 | 1.07 | 0.00 | 0.28 | 0.15 | 0.26 | 0.06 | 0.05 | 1.31 | 0.53 | 0.26 | 0.98 |
| B | 164 | Boul | 3 | SC | UN | 0.652 | 0.54 | 1.26 | 4.94 | 1.30 | 0.00 | 0.35 | 0.71 | 0.43 | 0.12 | 0.05 | 1.38 | 0.51 | 0.00 | 1.17 |
| C | 224 | Ney | 3 | SC | PT | 2.068 | 0.12 | 0.69 | 4.27 | 0.37 | 0.00 | 0.23 | 0.32 | 0.12 | 0.19 | 0.20 | 2.01 | 0.72 | 1.57 | 0.09 |
| C | 242 | Pil | 4 | SC | MT | 1.722 | 1.03 | 0.55 | 0.62 | 0.21 | 0.00 | 0.20 | 0.22 | 0.30 | 0.00 | 0.11 | 2.30 | 1.33 | 0.00 | 0.38 |

| | | | | | | | | | | | | | | | |
|---|---|---|---|---|---|---|---|---|---|---|---|---|---|---|---|
| - - - - Means Sq | -.--- | 1.59 | 1.33 | .686 | .140 | .910 | .515 | .559 | 1.84 | .170 | .405 | 2.38 | 1.65 | .779 | 3.93 |
| - - - - Means Ad | -.--- | .948 | 1.18 | 1.07 | .119 | .319 | .664 | .679 | 1.31 | .196 | .630 | 3.08 | 2.13 | .886 | 3.49 |
| - - - - Means SC | -.--- | .349 | .556 | 5.04 | 1.17 | .025 | .205 | .238 | .339 | .064 | .057 | 1.10 | .735 | .248 | .563 |
| - - - - varianc Sq | -.--- | .904 | 1.19 | .326 | .027 | 1.48 | .108 | .084 | 1.68 | .006 | .070 | 1.29 | .435 | .411 | 3.71 |
| - - - - varianc Ad | -.--- | .134 | .516 | .680 | .011 | .380 | .475 | .489 | 1.30 | .008 | .395 | 2.01 | .828 | .412 | 4.78 |
| - - - - varianc SC | -.--- | .097 | .137 | 4.77 | .736 | .005 | .023 | .052 | .118 | .003 | .004 | .514 | .064 | .260 | .133 |
| - - - - - P-values | -.--- | .001 | .108 | .000 | .000 | .077 | .106 | .123 | .014 | .005 | .017 | .004 | .000 | .079 | .000 |

TABLE 3

| | |
|---|---|
| group pat stg di | Initially thought larger in Adenocarcinoma |

| | | | | | | | | | | | | | | |
|---|---|---|---|---|---|---|---|---|---|---|---|---|---|---|
| gel ient | ag so | s43 | s29 | s36 | s40 | s42 | s76 | s53 | s83 | s92 | s94 | s84 | s100 | s96 |

| | | | | | | | | | | | | | | | | | | |
|---|---|---|---|---|---|---|---|---|---|---|---|---|---|---|---|---|---|---|
| A | 155 | Duc | 1 | Sq | PT | 0.08 | 0.26 | 0.08 | 0.91 | 0.08 | 0.50 | 0.09 | 0.41 | 0.02 | 0.92 | 0.74 | 0.21 | 1.15 |
| A | 143 | Mor | 3 | Sq | PT | 0.00 | 1.48 | 0.15 | 2.57 | 0.04 | 0.75 | 0.43 | 0.55 | 0.24 | 2.00 | 1.04 | 0.46 | 1.30 |
| A | 156 | Chi | 3 | Sq | MT | 0.07 | 0.36 | 0.12 | 1.81 | 0.04 | 0.95 | 1.50 | 0.44 | 0.57 | 1.38 | 1.36 | 0.44 | 0.90 |
| B | 180 | Cav | 1 | Sq | UN | 0.11 | 1.33 | 0.54 | 4.16 | 0.28 | 1.26 | 0.24 | 1.42 | 0.18 | 2.23 | 0.54 | 0.17 | 1.53 |
| B | 171 | Wae | 3 | Sq | UN | 0.03 | 2.09 | 0.19 | 0.83 | 0.05 | 0.95 | 0.14 | 0.27 | 0.00 | 0.53 | 0.79 | 0.10 | 2.13 |
| B | 179 | Dur | 3 | Sq | PT | 0.11 | 0.96 | 0.24 | 1.51 | 0.08 | 0.61 | 0.13 | 0.52 | 0.05 | 0.64 | 0.45 | 0.12 | 1.82 |
| C | 225 | Arc | 2 | Sq | UN | 0.12 | 2.04 | 0.51 | 3.70 | 0.21 | 0.52 | 0.63 | 1.87 | 0.54 | 6.11 | 0.65 | 0.12 | 3.42 |
| C | 235 | Por | 2 | Sq | UN | 0.04 | 1.48 | 0.24 | 2.32 | 0.09 | 0.49 | 0.65 | 1.06 | 0.98 | 3.67 | 0.56 | 0.02 | 2.33 |
| C | 237 | Guy | 3 | Sq | PT | 0.08 | 0.96 | 0.35 | 2.50 | 0.13 | 0.59 | 0.36 | 0.99 | 0.61 | 3.59 | 0.37 | 0.00 | 1.10 |
| A | 141 | Del | 1 | Ad | PT | 0.11 | 0.65 | 0.20 | 2.93 | 0.07 | 5.85 | 1.61 | 0.93 | 0.73 | 3.67 | 2.02 | 0.87 | 1.80 |
| A | 150 | Bog | 2 | Ad | PT | 0.06 | 0.76 | 0.34 | 4.72 | 0.16 | 1.43 | 2.70 | 1.16 | 0.81 | 4.46 | 1.40 | 0.37 | 2.67 |
| A | 144 | Des | 3 | Ad | PT | 0.15 | 6.85 | 0.26 | 2.96 | 0.08 | 3.72 | 2.08 | 0.64 | 0.41 | 3.24 | 2.09 | 0.60 | 4.41 |
| A | 158 | Suc | 3 | Ad | UN | 0.03 | 1.20 | 0.32 | 4.56 | 0.19 | 0.69 | 0.63 | 1.07 | 0.41 | 2.43 | 0.78 | 0.13 | 0.30 |
| B | 167 | Coul | 3 | Ad | PT | 0.06 | 1.52 | 0.19 | 1.26 | 0.15 | 0.39 | 0.17 | 0.26 | 0.00 | 0.51 | 0.14 | 0.00 | 1.40 |
| B | 172 | Bon | 3 | Ad | PT | 0.07 | 0.76 | 0.30 | 1.02 | 0.07 | 1.12 | 0.26 | 0.36 | 0.08 | 0.32 | 0.17 | 0.03 | 3.30 |
| C | 241 | Ber | 1 | Ad | UN | 0.12 | 2.01 | 0.37 | 3.94 | 0.13 | 0.42 | 0.52 | 1.33 | 0.59 | 7.94 | 0.93 | 0.14 | 3.30 |
| C | 234 | Dam | 3 | Ad | PT | 0.09 | 1.04 | 0.19 | 1.85 | 0.05 | 0.91 | 0.13 | 0.53 | 0.07 | 2.59 | 0.58 | 0.17 | 1.75 |
| A | 145 | Bai | 3 | SC | MT | 0.00 | 0.22 | 0.18 | 0.26 | 0.03 | 4.00 | 0.00 | 0.13 | 0.00 | 0.20 | 0.33 | 0.01 | 0.16 |
| A | 146 | Cha | 4 | SC | MT | 0.00 | 0.28 | 0.09 | 0.18 | 0.00 | 0.71 | 0.00 | 0.07 | 0.00 | 0.06 | 0.43 | 0.13 | 0.49 |
| A | 148 | Bri | 4 | SC | MT | 0.00 | 0.30 | 0.04 | 0.41 | 0.02 | 0.16 | 0.13 | 0.15 | 0.00 | 0.45 | 0.75 | 0.17 | 0.50 |
| A | 151 | Moy | 4 | SC | MT | 0.00 | 0.41 | 0.17 | 0.99 | 0.01 | 0.25 | 0.09 | 0.26 | 0.00 | 0.59 | 0.85 | 0.05 | 0.88 |
| A | 152 | Boua | 4 | SC | MT | 0.00 | 0.21 | 0.16 | 0.58 | 0.03 | 0.24 | 0.32 | 0.20 | 0.19 | 0.63 | 0.66 | 0.00 | 0.35 |
| A | 157 | Couc | 2 | SC | UN | 0.00 | 0.59 | 0.14 | 0.87 | 0.00 | 0.16 | 0.07 | 0.25 | 0.00 | 0.94 | 0.64 | 0.00 | 1.11 |
| B | 164 | Boul | 3 | SC | UN | 0.00 | 1.07 | 0.17 | 1.00 | 0.09 | 0.39 | 0.22 | 0.29 | 0.00 | 0.62 | 0.74 | 0.14 | 2.34 |
| C | 224 | Ney | 3 | SC | PT | 0.14 | 1.45 | 0.31 | 1.91 | 0.06 | 0.22 | 0.11 | 0.40 | 0.00 | 1.54 | 0.13 | 0.02 | 1.76 |
| C | 242 | Pil | 4 | SC | MT | 0.00 | 1.02 | 0.26 | 1.61 | 0.07 | 0.24 | 0.20 | 0.94 | 0.20 | 2.12 | 0.10 | 0.00 | 0.49 |

| | | | | | | | | | | | | | |
|---|---|---|---|---|---|---|---|---|---|---|---|---|---|
| - - - - Means Sq | .070 | 1.21 | .270 | 2.25 | .111 | .736 | .463 | .837 | .354 | 2.34 | .721 | .182 | 1.74 |
| - - - - Means Ad | .085 | 1.84 | .271 | 2.90 | .112 | 1.81 | 1.01 | .784 | .385 | 3.14 | 1.01 | .287 | 2.36 |
| - - - - Means SC | .016 | .617 | .168 | .870 | .033 | .706 | .125 | .297 | .043 | .793 | .513 | .058 | .898 |
| - - - - varianc Sq | .001 | .423 | .027 | 1.30 | .006 | .070 | .195 | .292 | .113 | 3.34 | .096 | .027 | .628 |
| - - - - varianc Ad | .001 | 4.28 | .005 | 2.06 | .002 | 3.79 | .968 | .155 | .096 | 5.81 | .579 | .092 | 1.69 |
| - - - - varianc SC | .002 | .205 | .006 | .350 | .000 | 1.55 | .011 | .067 | .007 | .430 | .076 | .004 | .526 |
| - - - - - P-values | .006 | .144 | .120 | .002 | .013 | .166 | .020 | .021 | .024 | .032 | .127 | .078 | .015 |

TABLE 4

| group pat stg di | | | | | | Initially thought larger in Small Cell except that 103 also big in Adenocarcinoma. | | | | | | | | | | | | | | |
|---|---|---|---|---|---|---|---|---|---|---|---|---|---|---|---|---|---|---|---|---|
| gel ient | | | | ag so | | s103 | s14 | s15 | s16 | s17 | s22 | s27 | s31 | s33 | s41 | s48 | 549 | s50 | s68 | s75 |
| A | 155 | Duc | 1 | Sq | PT | 0.18 | 0.30 | 0.38 | 0.59 | 0.23 | 0.15 | 0.00 | 0.10 | 0.15 | 0.06 | 0.39 | 0.09 | 0.06 | 0.02 | 0.30 |
| A | 143 | Mor | 3 | Sq | PT | 0.23 | 0.18 | 0.26 | 0.53 | 0.16 | 0.21 | 0.00 | 0.16 | 0.00 | 0.09 | 0.52 | 0.17 | 0.00 | 0.05 | 0.50 |
| A | 156 | Chi | 3 | Sq | MT | 0.00 | 0.24 | 0.11 | 0.30 | 0.04 | 0.07 | 0.00 | 0.00 | 0.00 | 0.06 | 0.14 | 0.12 | 0.01 | 0.09 | 0.28 |
| B | 180 | Cav | 1 | Sq | UN | 0.50 | 0.22 | 0.22 | 0.16 | 0.49 | 0.11 | 0.05 | 0.11 | 0.00 | 0.45 | 0.59 | 0.12 | 0.14 | 0.06 | 0.48 |
| B | 171 | Wae | 3 | Sq | UN | 0.20 | 0.14 | 0.16 | 0.14 | 0.12 | 0.09 | 0.08 | -.-- | 0.00 | 0.33 | 0.59 | 0.21 | 0.12 | 0.03 | 0.35 |
| B | 179 | Dur | 3 | Sq | PT | 0.39 | 0.25 | 0.31 | 0.16 | 0.80 | 0.15 | 0.07 | 0.10 | 0.06 | 0.17 | 0.63 | 0.11 | 0.06 | 0.03 | 0.41 |
| C | 225 | Arc | 2 | Sq | UN | 0.30 | 0.21 | 0.37 | 0.49 | 0.35 | 0.13 | 0.06 | 0.05 | 0.00 | 0.11 | 0.51 | 0.35 | 0.06 | 0.09 | 0.52 |
| C | 235 | Por | 2 | Sq | UN | 0.48 | 0.12 | 0.28 | 0.37 | 0.28 | 0.05 | 0.06 | 0.10 | 0.00 | 0.06 | 0.38 | 0.18 | 0.10 | 0.01 | 0.42 |
| C | 237 | Guy | 3 | Sq | PT | 0.12 | 0.06 | 0.05 | 0.54 | 0.22 | 0.10 | 0.03 | 0.00 | 0.00 | 0.10 | 0.56 | 0.00 | 0.00 | 0.03 | 0.35 |
| A | 141 | Del | 1 | Ad | PT | 0.79 | 0.26 | 0.48 | 0.33 | 0.09 | 0.19 | 0.03 | 0.10 | 0.00 | 0.07 | 0.22 | 0.18 | 0.12 | 0.06 | 0.51 |
| A | 150 | Bog | 2 | Ad | PT | 0.49 | 0.31 | 0.40 | 1.01 | 0.08 | 0.06 | 0.00 | 0.17 | 0.00 | 0.25 | 0.26 | 0.21 | 0.06 | 0.07 | 0.49 |
| A | 144 | Des | 3 | Ad | PT | 0.59 | 0.35 | 0.26 | 0.78 | 0.00 | 0.21 | 0.00 | 0.15 | 0.00 | 0.34 | 0.27 | 0.25 | 0.06 | 0.10 | 0.39 |
| A | 158 | Suc | 3 | Ad | UN | 0.34 | 0.12 | 0.12 | 0.19 | 0.14 | 0.14 | 0.04 | 0.07 | 0.00 | 0.11 | 0.36 | 0.13 | 0.03 | 0.18 | 0.38 |
| B | 167 | Cou | 3 | Ad | PT | 0.32 | 0.00 | 0.11 | 0.03 | 0.57 | 0.18 | 0.00 | 0.12 | 0.00 | 0.16 | 0.46 | 0.28 | 0.50 | 0.07 | 0.40 |
| B | 172 | Bon | 3 | Ad | PT | 0.39 | 0.22 | 0.08 | 0.20 | 0.35 | 0.07 | 0.00 | 0.13 | 0.00 | 0.36 | 0.42 | 0.57 | 0.02 | 0.05 | 0.47 |
| C | 241 | Ber | 1 | Ad | UN | 0.43 | 0.37 | 0.10 | 1.16 | 0.05 | 0.11 | 0.18 | 0.39 | 0.00 | 0.12 | 0.32 | 0.31 | 0.05 | 0.17 | 0.60 |
| C | 234 | Dam | 3 | Ad | PT | 0.25 | 0.13 | 0.18 | 0.35 | 0.34 | 0.08 | 0.03 | 0.23 | 0.00 | 0.09 | -.-- | 0.00 | 0.07 | 0.08 | 0.42 |
| A | 145 | Bai | 3 | SC | MT | 0.10 | 0.47 | 0.64 | 1.06 | 0.53 | 0.13 | 0.12 | 0.50 | 0.16 | 0.33 | 0.22 | 0.04 | 0.35 | 0.14 | 0.30 |
| A | 146 | Cha | 4 | SC | MT | 0.02 | 0.83 | 1.10 | 1.84 | 0.96 | 0.42 | 0.21 | 0.32 | 0.21 | 0.44 | 0.61 | 0.11 | 1.59 | 0.20 | 0.56 |
| A | 148 | Bri | 4 | SC | MT | 0.24 | 2.35 | 2.67 | 1.95 | 0.83 | 0.44 | 0.29 | 0.50 | 0.27 | 0.35 | 0.54 | 0.27 | 0.87 | 0.28 | 0.84 |
| A | 151 | Moy | 4 | SC | MT | 0.45 | 0.86 | 1.28 | 0.60 | 0.68 | 0.39 | 0.22 | 0.49 | 0.18 | 0.13 | 0.49 | 0.35 | 0.56 | 0.16 | 0.41 |
| A | 152 | Boua | 4 | SC | MT | 0.79 | 0.70 | 3.31 | 1.15 | 0.75 | 0.25 | 0.23 | 1.00 | 0.11 | 0.23 | 0.23 | 0.23 | 0.36 | 0.14 | 0.32 |
| A | 157 | Couc | 2 | SC | UN | 0.32 | 0.21 | 0.48 | 1.22 | 0.63 | 0.27 | 0.10 | 0.39 | 0.05 | 0.08 | 0.57 | 0.52 | 0.44 | 0.27 | 0.60 |
| B | 164 | Boul | 3 | SC | UN | 0.53 | 0.67 | 0.46 | 0.29 | 1.23 | 0.12 | 0.44 | 0.45 | 0.00 | 0.32 | 0.64 | 0.36 | 0.44 | 0.15 | 0.49 |
| C | 224 | Ney | 3 | SC | PT | 0.22 | 1.94 | 1.82 | 0.44 | 1.59 | 0.37 | 0.25 | 0.33 | 0.07 | 0.21 | 0.49 | 0.05 | 0.56 | 0.13 | 0.92 |
| C | 242 | Pil | 4 | SC | MT | 0.02 | 0.20 | 0.48 | 0.83 | 0.67 | 0.04 | 0.09 | 0.12 | 0.00 | 0.16 | 0.55 | 0.29 | 0.75 | 0.27 | 0.86 |

| | | | | | | | | | | | | | | | |
|---|---|---|---|---|---|---|---|---|---|---|---|---|---|---|---|
| - - - - Means Sq | .266 | .188 | .239 | .364 | .299 | .118 | .039 | .077 | .023 | .157 | .477 | .150 | .061 | .045 | .400 |
| - - - - Means Ad | .449 | .220 | .216 | .504 | .202 | .130 | .034 | .169 | .000 | .187 | .328 | .242 | .114 | .097 | .455 |
| - - - - Means SC | .521 | .915 | 1.35 | 1.04 | .874 | .271 | .215 | .456 | .117 | .249 | .483 | .245 | .658 | .191 | .588 |
| - - - - varianc Sq | .027 | .005 | .012 | .032 | .052 | .002 | .001 | .003 | .002 | .018 | .023 | .009 | .002 | .000 | .007 |
| - - - - varianc Ad | .030 | .016 | .022 | .177 | .039 | .003 | .003 | .010 | .000 | .013 | .007 | .027 | .024 | .002 | .005 |
| - - - - varianc SC | .120 | .556 | 1.08 | .335 | .114 | .021 | .011 | .056 | .009 | .013 | .023 | .024 | .153 | .003 | .055 |
| - - - - - P-values | .099 | .003 | .000 | .006 | .000 | .004 | .000 | .000 | .002 | .297 | .067 | .299 | .000 | .000 | .046 |

TABLE 5

| group pat stg di | | | | | | Initially thought larger in Small Cell. | | | | | | | | | | | | | | | |
|---|---|---|---|---|---|---|---|---|---|---|---|---|---|---|---|---|---|---|---|---|---|
| gel ient | | | | ag so | | s47 | s57 | s58 | s59 | s61 | s66 | s67 | s73 | s74 | s81 | s105 | s85 | s86 | s97 | s98 | s106 |
| A | 155 | Duc | 1 | Sq | PT | 0.29 | 0.60 | 0.71 | 1.35 | 0.32 | 0.00 | 0.08 | 0.09 | 0.11 | 0.10 | 0.46 | 0.31 | 0.97 | 0.22 | 0.58 | 0.52 |
| A | 143 | Mor | 3 | Sq | PT | 0.17 | 0.31 | 0.33 | 0.84 | 0.00 | 0.00 | 0.41 | 0.05 | 0.27 | 0.11 | 0.30 | 0.34 | 0.46 | 0.34 | 0.42 | 0.46 |
| A | 156 | Chi | 3 | Sq | MT | 0.00 | 0.14 | 0.18 | 0.34 | 0.00 | 0.31 | 0.04 | 0.00 | 0.15 | 0.00 | 0.43 | 0.22 | 0.00 | 0.00 | 0.43 | 0.41 |
| B | 180 | Cav | 1 | Sq | UN | 0.20 | 0.51 | 0.78 | 2.03 | 0.23 | 0.28 | 0.10 | 0.09 | 0.09 | 0.10 | 0.34 | 0.73 | 0.32 | 0.55 | 0.16 | 0.30 |
| B | 171 | Wae | 3 | Sq | UN | 0.15 | 0.46 | 0.68 | 0.93 | 0.14 | 0.27 | 0.07 | 0.00 | 0.00 | 0.27 | 0.20 | 0.15 | 0.35 | 0.71 | 0.30 | 0.55 |
| B | 179 | Dur | 3 | Sq | PT | 0.15 | 0.73 | 1.64 | 2.35 | 0.29 | 0.64 | 1.36 | 0.04 | 0.13 | 0.41 | 0.37 | 0.22 | 0.48 | 0.40 | 0.35 | 0.34 |
| C | 225 | Arc | 2 | Sq | UN | 0.11 | 0.40 | 0.46 | 0.85 | 0.07 | 0.23 | 0.27 | 0.08 | 0.15 | 0.00 | 0.28 | 1.53 | 0.23 | 0.57 | 0.36 | 0.44 |
| C | 235 | Por | 2 | Sq | UN | 0.14 | 0.49 | 0.32 | 1.58 | 0.07 | 0.34 | 0.04 | 0.00 | 0.30 | 0.10 | 0.20 | 0.19 | 0.19 | 1.18 | 0.41 | 0.24 |
| C | 237 | Guy | 3 | Sq | PT | 0.16 | 0.27 | 0.44 | 1.04 | 0.06 | 0.21 | 0.05 | 0.07 | 0.19 | 0.00 | 0.15 | 0.21 | 0.38 | 0.27 | 0.30 | 0.15 |
| A | 141 | Del | 1 | Ad | PT | 0.16 | 0.40 | 0.34 | 0.94 | 0.00 | 0.00 | 0.43 | 0.15 | 0.36 | 0.00 | 0.52 | 0.64 | 0.50 | 0.43 | 0.45 | 0.24 |
| A | 150 | Bog | 2 | Ad | PT | 0.07 | 0.54 | 0.47 | 0.55 | 0.00 | 0.19 | 0.19 | 0.13 | 0.00 | 0.00 | 0.44 | 0.43 | 0.15 | 0.43 | 0.33 | 0.25 |
| A | 144 | Des | 3 | Ad | PT | 0.10 | 0.34 | 0.17 | 0.35 | 0.00 | 0.38 | 0.66 | 0.00 | 0.24 | 0.00 | 0.75 | 0.26 | 0.16 | 0.52 | 0.64 | 0.64 |
| A | 158 | Suc | 3 | Ad | UN | 0.12 | 0.40 | 0.65 | 0.91 | 0.00 | 0.48 | 0.09 | 0.25 | 0.19 | 0.00 | 0.30 | 2.92 | -.-- | 0.43 | 0.11 | 0.19 |
| B | 167 | Coul | 3 | Ad | PT | 0.12 | 0.41 | 0.77 | 1.82 | 0.15 | 0.58 | 0.11 | 0.08 | 0.00 | 0.17 | 0.35 | 0.59 | 0.24 | 0.17 | 0.13 | 0.29 |
| B | 172 | Bon | 3 | Ad | PT | 0.25 | 0.34 | 0.46 | 1.19 | 0.40 | 0.31 | 0.02 | 0.00 | 0.00 | 0.76 | 0.25 | 0.19 | 0.11 | 0.07 | 0.19 | 0.29 |
| C | 241 | Ber | 1 | Ad | UN | 0.42 | 0.36 | 0.48 | 1.19 | 0.16 | 0.05 | 0.07 | 0.11 | 0.20 | 0.12 | 0.17 | 0.32 | 0.44 | 0.50 | 0.62 | 0.55 |
| C | 234 | Dam | 3 | Ad | PT | 0.15 | 0.27 | 0.39 | 1.57 | 0.10 | 0.03 | 0.05 | 0.04 | 0.08 | 0.00 | 0.23 | 0.32 | 0.24 | 0.45 | 0.49 | 0.31 |
| A | 145 | Bai | 3 | SC | MT | 0.33 | 0.38 | 0.74 | 1.35 | 0.68 | 0.60 | 1.32 | 0.16 | 0.32 | 0.21 | 0.45 | 0.35 | 0.76 | 0.17 | 1.50 | 0.88 |
| A | 146 | Cha | 4 | SC | MT | 0.51 | 1.54 | 1.34 | 3.75 | 1.43 | 1.17 | 0.07 | 0.28 | 0.82 | 0.74 | 0.83 | 1.27 | 1.01 | 1.74 | 0.59 | 0.88 |
| A | 148 | Bri | 4 | SC | MT | 0.51 | 0.95 | 1.10 | 2.84 | 1.38 | 2.14 | 1.50 | 0.44 | 0.43 | 0.55 | 1.15 | 1.31 | 1.98 | 1.65 | 1.09 | 1.56 |
| A | 151 | Moy | 4 | SC | MT | 0.47 | 1.53 | 1.36 | 2.40 | 1.03 | 0.74 | 1.95 | 0.21 | 0.48 | 1.03 | 0.88 | 0.66 | 1.92 | 1.33 | 1.16 | 0.92 |
| A | 152 | Boua | 4 | SC | MT | 0.34 | 0.74 | 1.39 | 3.39 | 0.27 | 1.07 | 1.46 | 0.25 | 0.43 | 0.74 | 1.08 | 0.49 | 0.45 | 0.16 | 0.86 | 0.95 |
| A | 157 | Couc | 2 | SC | UN | 0.39 | 0.87 | 0.69 | 2.46 | 0.63 | 0.50 | 1.30 | 0.19 | 0.00 | 0.20 | 0.47 | 0.67 | 1.25 | 0.69 | 1.09 | 0.38 |
| B | 164 | Boul | 3 | SC | UN | 0.40 | 0.66 | 1.42 | 2.47 | 0.45 | 0.77 | 1.23 | 0.08 | 0.24 | 0.21 | 0.39 | 0.29 | 1.35 | 0.86 | 0.43 | 0.96 |
| C | 224 | Ney | 3 | SC | PT | 0.32 | 0.74 | 1.11 | 1.36 | 0.07 | 0.25 | 0.81 | 0.12 | 0.15 | 0.30 | 0.34 | 0.37 | 0.47 | 0.63 | 0.21 | 0.59 |
| C | 242 | Pil | 4 | SC | MT | 0.21 | 0.94 | 0.66 | 1.91 | 0.10 | 0.51 | 0.14 | 0.15 | 0.12 | 0.21 | 0.33 | 0.07 | 0.36 | 0.63 | 0.34 | 0.27 |

| | | | | | | | | | | | | | | | | |
|---|---|---|---|---|---|---|---|---|---|---|---|---|---|---|---|---|
| - - - - Means Sq | .151 | .435 | .616 | 1.25 | .132 | .253 | .268 | .046 | .154 | .121 | .302 | .434 | .375 | .470 | .367 | .380 |
| - - - - Means Ad | .175 | .382 | .466 | 1.06 | .100 | .252 | .203 | .095 | .134 | .130 | .376 | .710 | .263 | .374 | .369 | .345 |
| - - - - Means SC | .385 | .929 | 1.09 | 2.43 | .672 | .860 | 1.08 | .209 | .332 | .465 | .657 | .610 | 1.06 | .872 | .808 | .819 |
| - - - - varianc Sq | .005 | .032 | .189 | .405 | .014 | .036 | .183 | .001 | .008 | .018 | .011 | .200 | .070 | .116 | .012 | .017 |
| - - - - varianc Ad | .012 | .006 | .033 | .238 | .019 | .047 | .050 | .007 | .018 | .069 | .035 | .825 | .023 | .026 | .045 | .025 |
| - - - - varianc SC | .009 | .148 | .100 | .677 | .265 | .312 | .397 | .011 | .060 | .096 | .106 | .183 | .374 | .341 | .190 | .145 |
| - - - - - P-values | .000 | .000 | .001 | .000 | .001 | .002 | .000 | .001 | .046 | .010 | .007 | .652 | .001 | .042 | .005 | .001 |

1. A protein which is overexpressed in lung tumors compared to non-tumor tissue selected from the group consisting of Spot 14, 15, 16, 17, 21, 22, 27, 29, 31, 33, 40, 42, 43, 47, 50, 53, 57, 58, 59, 61, 62, 66, 68, 73, 74, 79, 81, 83, 84, 86, 90, 94, 95, 96, 97, 98, 100, 101, 102, 105 and 106.

2. An antibody or antigen binding fragment thereof which specifically binds a protein of claim 1.

3. A method of screening for, establishing subtype of, or monitoring the progression of lung tumor comprising:
a) determining an amount of at least one protein selected from the group consisting of Spot 14, 15, 16, 17, 21, 22, 27, 29, 31, 33, 40, 42, 43, 47, 50, 53, 57, 58, 59, 61, 62, 66, 67, 68, 73, 74, 79, 80, 81, 83, 84, 86, 90, 92, 94, 95, 96, 97, 98, 100, 101, 102, 105, 106, 107 and 109 in an animal or human or in a sample from an animal or human; and
b) correlating the amount with the presence, subtype, or stage of lung tumor.

4. The method of claim 3 wherein the amount of said protein is determined with an immunological assay.

5. The method of claim 3 wherein the amount of said protein is determined with 2-D gel electrophoresis.

6. The method of claim 3 wherein a plurality of proteins is selected from the group consisting of Spot 14, 15, 16, 17, 21, 22, 27, 29, 31, 33, 40, 42, 43, 47, 50, 53, 57, 58, 59, 61, 62, 66, 67, 68, 73, 79, 80, 81, 83, 84, 86, 90, 92, 94, 95, 96, 97, 98, 100, 101, 102, 105, 106, 107 and 109 in an animal or human or in a sample from an animal or human.

7. The method of claim 6 wherein the amounts of proteins are determined with an immunological assay.

8. The method of claim 6 wherein the amounts of proteins are determined with 2-C gel electrophoresis.

9. A method of making an antibody or antigen binding fragment thereof which specifically binds a protein of claim 1, comprising:
a) immunizing an animal with a protein of claim 1;
b) collecting serum from said animal; and
c) isolating an antibody or antigen binding fragment which specifically binds a protein of claim 1 from the serum.

10. A method of making a monoclonal antibody or antigen binding fragment thereof which specifically binds a protein of claim 1, comprising:
a) immunizing an animal with a protein of claim 1;
b) isolating splenocytes from the animal;
c) fusing said splenocytes with myeloma cells;
d) growing the fused cells;
e) testing the fused cells for antibodies which specifically bind a protein of claim 1; and
f) isolating any antibody or an antigen binding fragment which specifically binds a protein of claim 1.

11. A method of detecting tumor tissue in a tissue section comprising:
a) treating a tissue section with an antibody specific for an epitope formed by heterodimerization of MRP8 and MRP14;
b) washing away any unbound antibody; and
c) determining the amount of bound antibody in the tissue section as an indication of the presence of tumor tissue.

12. The method of claim 11 wherein the tumor is a lung tumor.

13. A method of detecting a tumor in an animal or human comprising:
a) separating proteins in a serum sample from said animal or human;
b) transferring said proteins to a membrane;
c) probing said proteins with an antibody specific for an epitope formed by heterodimerization of MRP8 and MRP14;
d) determining the amount of bound antibody;
e) integrating the intensity of reactivity in a band; and
f) correlating the integrated intensity with the presence or stage of tumor.

14. The method of claim 13 wherein the tumor is a lung tumor.

15. The method of claim 14 wherein the said ban is 14 kDa.

16. An isolated gene encoding for a protein of claim 1, wherein said protein comprises an amino acid sequence selected from the group consisting of
a) Seq. ID No. 1;
b) Seq. ID No. 2;
c) Seq. ID No. 5;
d) Seq. ID No. 6; and
e) Seq. ID No. 8.

17. A method of treating tumor in an animal or human in need thereof comprising:
a) conjugating the antibody or antigen binding fragment thereof as described in claim 2 with a radioactive substance, toxin or anti-tumor drug; and
b) administering an effective amount of the conjugate into said animal or human.

18. A method of treating tumor in an animal or human in need thereof comprising:
a) exposing immunocompetent cells from the animal or human to at least one protein selected from the group consisting of Spot 14, 15, 16, 17, 21, 22, 27, 29, 31, 33, 40, 42, 43, 47, 50, 53, 57, 58, 59, 61, 62, 66, 67, 68, 73, 74, 79, 80, 81, 83, 84, 86, 90, 92, 94, 95, 96, 97, 98, 100, 101, 102, 105, 106, 107 and 109; and
b) injecting said immunocompetent cells into the animal or human to treat a tumor.

19. The method of claim 3 wherein said at least one protein is Spot 107.

20. The method of claim 19 wherein said at least one protein is in a sample from an animal or human.
